(19) United States
(12) Patent Application Publication
(10) Pub. No.: US 2026/0031202 A1
(43) Pub. Date: Jan. 29, 2026

(54) COMPUTER-IMPLEMENTED SYSTEM AND METHOD FOR SECURE FEDERATED STUDIES

(71) Applicant: EVIDENTLI PTY LTD, Surry Hills (AU)

(72) Inventors: Guy TSAFNAT, Surry Hills (AU); Dean TSAFNAT, Surry Hills (AU)

(73) Assignee: EVIDENTLI PTY LTD, Surry Hills (AU)

(21) Appl. No.: 19/349,962

(22) Filed: Oct. 4, 2025

Related U.S. Application Data
(63) Continuation of application No. PCT/AU2024/050332, filed on Apr. 5, 2024.

(30) Foreign Application Priority Data
Apr. 5, 2023 (AU) 2023900994

(51) Int. Cl. G16H 10/20; G16H 15/00

(52) U.S. Cl. CPC G16H 10/20; G16H 15/00

(57) ABSTRACT

A computer-implemented federated studies system and method are disclosed. The system includes: a predefined common data model associated with a study; a set of sites of origin, each associated with a set of input devices for acquiring study data from subjects and a study data device. Each study data device includes: a database for storing study data derived from input devices; a data model translator for translating study data to a common data model format; a common model database for storing, in the data model, individual participant values derived from study data; a study compute module for determining site aggregate values from individual participant values; and an aggregate database for storing site aggregate values. A site of analysis is associated with a study analysis device that includes: an analysis compute module for processing site aggregate values received from sites of origin; and a reporting module for generating study reports.

### RELATED APPLICATION

This application is a continuation of International Application PCT/AU2024/050332 filed 5 Apr. 2024, which claims the benefit of Australian Provisional Patent Application No. 2023900994 titled “COMPUTER-IMPLEMENTED SYSTEM AND METHOD FOR SECURE FEDERATED STUDIES” and filed 5 Apr. 2023 in the name of Evidentli Pty Ltd, the entire contents of both of which are incorporated herein by reference as if fully set forth herein.

### TECHNICAL FIELD

The present disclosure relates to a system and associated methods for reliable and secure federated studies. In particular, the present disclosure relates to a computer-implemented system and related methods for use in reliable and secure federated studies in relation to clinical studies, observational studies, and other research activities.

### BACKGROUND

Federated studies are clinical trials, observational studies, and other types of research activities that are conducted across multiple physical locations (sites), such as hospitals, laboratories, universities, research institutions, and the like. The sites at which studies are performed are referred to herein as sites of origin and a site at which analysis of the data is performed is referred to herein as a site of analysis. A location may be both a site of origin and a site of analysis.

The advantages of federated studies include, but are not limited to:
the participants in a federated study represent a larger sample of the population than any one site could,
the logistics of conducting each study are simpler than conducting one large study, and
the heterogeneity of sites themselves represents a larger sample of sites.

In a federated study, each site of origin independently conducts a version of the study, and data from all sites of origin are analysed together at a site of analysis to produce a combined result. While there are several existing methods for conducting federated studies, each has limitations that prevent that method from being universally adopted. For example, different jurisdictions may define diagnoses differently or have utilise different parameters or ranges in relation to diagnoses or conditions.

Regardless of the mathematical methods used to combine results from multiple sites in a federated study, every federated study can potentially have a problem with consistency across the study sites. Variation in data production and collection, and materials used can differ and thus produce unexplained inaccuracies in the study results. Further, non-technical restrictions may affect the collation of data, such as jurisdictional legal requirements regarding privacy and data transmission.

Thus, a need exists to provide an improved system for conducting federated studies.

### SUMMARY

The present disclosure relates to a computer-implemented system and associated methods to conduct federated studies.

A first aspect of the present disclosure provides a computer-implemented federated studies system comprising:
a predefined common data model associated with a study;
a set of sites of origin, each site of origin being associated with a set of input devices for acquiring study data from subjects and a study data device, wherein each study data device includes:
   a native database for storing study data associated with the respective site of origin and derived from said set of input devices;
   a data model translator for translating stored study data to a format corresponding to said common data model;
   a common model database for storing. in said common data model, individual participant values derived from said study data;
   a study compute module for determining site aggregate values from said individual participant values; and
   an aggregate database for storing said site aggregate values; and
a site of analysis coupled to each site of origin via a communications network, wherein said site of analysis is associated with a study analysis device that includes:
   an analysis compute module for processing, in accordance with a predefined analysis profile, site aggregate values received from said sites of origin; and
   a reporting module for generating study reports based on said processed site aggregate values;
a central repository for storing and retrieving a set of data translation elements for managing extensions and mappings within study data sets associated with each site of origin; and
an Identifier Issuing Service configured to interface between each site of origin and said central repository to ensure elements within data sets having the same semantic meaning have a common unique identifier.

A second aspect of the present disclosure provides a method of conducting a federated study across a plurality of sites of origin and using a site of analysis, the method comprising the steps of:
defining a common data model for the study;
defining a set of analysis instructions for the study;
at each site of origin:
   acquiring, utilising least one input device, study data from a set of subjects;
   translating said study data to generate individual participant values stored in said common data model;
   processing said individual participant values to generate a set of site aggregate values;
   transmitting said site aggregate values to said site of analysis; and
   processing study aggregate values received from said Site of Analysis in accordance with said defined set of analysis instructions; and
at said site of analysis:
   processing site aggregate values received from at least one of said sites of origin in accordance with said defined set of analysis instructions; and
   generating a report based on said processed site aggregate values;
wherein the method unifies data translations across said plurality of Sites of Origin by:
   storing in a central repository a set of data translation elements for managing extensions and mappings within the federated study;
   on receipt of a request for a new extension, an Identifier Issuing Service communicating with said central repository to determine whether the requested extension exists;
   when the requested extension exists, the Identifier Issuing Service retrieving the requested extension from the central repository and forwarding the requested extension to the requesting translation client, and
   when the requested extension does not exist, the Identifier Issuing Service:
      issuing a new unique identifier for the requested extension,
      forwarding the new identifier for the requested extension to the requesting translation client, and
      forwarding the new identifier for the requested extension to the central repository for storage.

According to another aspect, the present disclosure provides an apparatus for implementing any one of the aforementioned methods. According to another aspect, the present disclosure provides a computer program product including a computer readable medium having recorded thereon a computer program that when executed on a processor of a computer implements any one of the methods described above.

Other aspects of the present disclosure are also provided.

### BRIEF DESCRIPTION OF THE DRAWINGS

One or more embodiments of the present disclosure will now be described by way of specific example(s) with reference to the accompanying drawings, in which:

FIG. 1 is a schematic block diagram representation of a computer-implemented federated study system in accordance with an embodiment of the present disclosure;

FIG. 2 is a schematic block diagram representation of a Site of Analysis in accordance with an embodiment of the present disclosure;

FIG. 3 is a schematic block diagram representation of a Site of Origin in accordance with an embodiment of the present disclosure;

FIG. 4 is a schematic block diagram representation of a federated studies system on which one or more embodiments of the present disclosure may be practised;

FIG. 5 is a schematic block diagram representation of a system that includes a general purpose computer on which one or more embodiments of the present disclosure may be practised;

FIG. 6 is a schematic block diagram representation of a system that includes a general smartphone on which one or more embodiments of the present disclosure may be practised;

FIG. 7 is a sequence diagram illustrating an exemplar practice workflow of a federated study performed by an embodiment of the system of the present disclosure;

FIG. 8 is a schematic block diagram representation of a site of origin implemented utilising a cloud-based computer architecture;

FIG. 9 is a schematic block diagram representation of a federated studies system implemented utilising a cloud-based computer architecture;

FIG. 10 is a schematic block diagram representation of a federated studies system implemented utilising a cloud-based computer architecture;

FIG. 11 is a schematic block diagram representation of a federated studies system implemented utilising a cloud-based computer architecture; and

FIG. 12 is a schematic block diagram representation illustrating a portion of a federated studies network having a Site of Origin, an Identifier Issuing Service, and a Central Repository.

Method steps or features in the accompanying drawings that have the same reference numerals are to be considered to have the same function(s) or operation(s), unless the contrary intention is expressed or implied.

### Glossary

### Site of Origin

A site that holds sensitive data about individuals that are to be used in a research project without transmitting that sensitive data outside the site. This description refers to multiple sites of origin that are labelled herein as Site₁, Site₂ and so on up to Siteₛ. More generally, each site is referred to as Siteᵢ.

### Site of Analysis

The site of analysis is labelled herein as Site_{A}. The Site of Analysis may coincide (be co-located or integral) with one of the sites of origin or may be a separate, discrete entity.

### Individual Participant Value: Yⱼ

One or more numerical values about one person (person j), that could potentially be used to identify or expose sensitive information about person j.

### Aggregate Value: Y

A data element that contains information about a cohort of people and that cannot be used to identify or expose sensitive information about a person or persons. Aggregate Values are safe to transmit between sites. Examples of Aggregate Values are counts of participants and averages of Individual Participant Values. Depending on the particular scenario and implementation, Aggregate Values may be exchanged back and forth between sites multiple times in the course of a single federated study.

### Site Aggregate: Ŷᵢ

An Aggregate Value that pertains to information available for one site of origin. The symbol for a Site Aggregate is followed by a subscript i, for example xᵢ. An example of a Site Aggregate is the number of participants at the site (nᵢ).

### Study Aggregate: Ŷ

An Aggregate Value that pertains to information available for an entire study. The symbol for a Study Aggregate is not followed by a subscript, for example xᵢ. An example of a Study Aggregate is the number of participants in the entire study (N).

### Task

A Study Aggregate that Site_{A} is tasked with calculating (i.e., a formula that needs site data to be calculated).

### Number of Study Participants: N

A special Study Aggregate that is the number of participants in the entire study.

### Number of Site Participants: nᵢ

A special Site Aggregate that is the number of participants in Siteᵢ.

### Number of Sites: s

The total number of sites of origin participating in a study.

### DETAILED DESCRIPTION

The present disclosure provides a computer-implemented system and associated methods for conducting federated studies. The system includes a set of sites of origin and a set of sites of analysis. In some embodiments, the sites of analysis are separate and discrete from the sites of origin, wherein each site of origin is coupled to a site of analysis via a communications link to enable the transmission of data from each site of origin to the respective site of analysis. In other embodiments, one or more of the sites of analysis are co-located or integral with a site of origin, with one or more communications links enabling transmission of data among sites of origin and a site of analysis in instances where a site of analysis is not co-located or integral with a site of origin.

A base embodiment utilises a single site of analysis. Other embodiments may utilise a plurality of sites of analysis. For example, one embodiment relates to a network scenario in which there are hierarchical subnetworks of sites of origin and sites of analysis, wherein those sites of analysis feed data to a central site of analysis. Such an hierarchical arrangement provides a cascading set of sites of origin and sites of analysis.

One implementation may relate, for example, to a central site of analysis administered by a pharmaceutical company. Subnetworks relate to different health districts that administer district sites of analysis, with each district site of analysis receiving data from hospitals associated with that health district. In such an implementation, the district sites of analysis become sites of origin for the central site of analysis.

For the sake of clarity, embodiments described herein will relate generally to a single hierarchical level in which a set of sites of origin reports to a single site of analysis. However, it will be appreciated by a person skilled in the art that the scope of the invention described herein covers alternative embodiments having multiple levels of analysis.

For the sake of clarity, embodiments described herein assume an embodiment of simple network connections. However, it will be appreciated by a person skilled in the art that the scope of the invention described herein covers alternative embodiments of networks that may include, for example, transmission (e.g., routers), security (e.g., firewalls) and acceleration (e.g., cache servers), and the like.

Existing approaches to federated studies encounter a technical difficulty of capturing data consistently across different sites of origin such that the data can be processed in a consistent manner. Further, there are technical difficulties in ensuring anonymity of personal data collected from different sites of origin.

The system of the present disclosure enforces data compatibility across the sites of origin to ensure reliability of results. In particular, the system includes one or more steps that transfer data from multiple sources to be aggregated and standardised in accordance with a predefined common data model. In some embodiments, the transfer of data may include translation, where required. In some embodiments, the system utilises intelligent data ingress that enables data from multiple sources to be aggregated and standardised in accordance with a predefined common data model. A data model conforms with the predefined common data model if that data model can be used by a site of origin to generate a data aggregate that is understandable by the site of analysis.

In some embodiments, the sites of origin and the site of analysis have a coordinated common data model. The coordinated common data model may be predefined by an administrator of a study to be performed, or otherwise coordinated among the various sites of origin and site of analysis. In such embodiments, data transmitted from a site of origin to the site of analysis has been prepared by the respective site of origin to conform with the coordinated common data model. In alternative embodiments, each site of origin uses a data model, which may or may not conform with the coordinated common data model, and the site of analysis has a translation service that can translate, where necessary, the aggregate information from each site of origin into a common data model.

In some embodiments, the common data model is the Observational Medical Outcomes Partnership Common Data Model (OMOP CDM or OMOP for short), Oracle Health Foundations, National Patient-Centered Clinical Research Network (PCORNet), Sentinel, and Informatics for Integrating Biology & the Bedside (I2B2), or a user-defined data model, may equally be utilised.

In some embodiments, the system provides data governance that enforces and audits data access permissions. The system does not transmit data relating to individual participants, thus addressing privacy and legal concerns.

In some embodiments, the system provides a user interface in the form of a dashboard that provides users with self-serve clinical analytics, enabling users of different technical proficiency to interrogate research data and generate reports.

### Data Pooling

Collecting data from the sites where data are recorded, and transmitting these data records to a site where the data records are to be analysed, is called “Data Pooling”. Data pooling is considered the current standard for calculating the most reliable results of federated studies. However, the transmission of Individual Participant Data between sites poses a risk to patient privacy and may be subject to legal controls.

Notably, the limitation here is not confined to the cryptographic protection of the data in transit and/or at rest, nor the reliable removal of personally identifying information from the data. Rather, the constraint is that most jurisdictions have legal restrictions that prohibit data transmission and storage and these non-technical restrictions are impossible to solve just using technologies such as encryption. Consequently, data pooling is often limited to a single site, which may prevent statistically useful sample sizes.

### Meta-Analysis

Meta-analysis is a method that avoids having to send Individual Participant Data and alleviates the risk to participant privacy by only transmitting aggregated information calculated at each site. These aggregates are combined at a common site that produces the study result. However, for most values these calculations can only approximate the results that would be achieved by data pooling.

There are several approximation techniques used to combine site data in meta-analysis techniques. Each approximation technique makes certain assumptions and often these assumptions are not stated explicitly. Not including the assumptions has a number of adverse consequences, including: (i) making it harder for the reader to understand the report; failing to convey the intentions of the author; and/or (iii) forcing the reader to infer these assumptions from the methods used, which is prone to error. Failing to state the assumptions associated with approximation techniques may result in a reader interpreting approximations as exact values, which is a mistake.

### Data Aggregates

Some approaches to conducting federated studies utilise data aggregates from different study sites. Examples of data aggregates are the number of participants who died during the study period, the median length of stay of a cohort of participants, and the absolute risk reduction between two study arms. Data aggregates are easy to calculate at every site in which data resides, and are transmitted from the sites with the original data (sites of origin), in lieu of sensitive data, to the remote site in which analysis of the data is conducted (site of analysis).

Some aggregate data can be calculated exactly at the site of analysis with results that are exactly equivalent to the results that would have been obtained from data pooling. For example, to know the total number of patients in the study, the number of patients from each site can be added together.

Other types of aggregate data cannot be calculated exactly from other data aggregates. For example, to exactly calculate the absolute risk reduction between two experimental groups, the average for each site is calculated and sent to the site of analysis. To calculate the average absolute risk reduction across the whole study population, certain assumptions have to be made. For example, the absolute risk reduction for each site should be weighted according to the number of participants at each site, or more commonly, by the homogeneity of patients at each site. With these assumptions, the calculated absolute risk reduction is only an approximation of the value that would have been obtained from data pooling.

### Computer-Implemented System and Associated Method for Conducting Federated Studies

Regardless of the mathematical methods used to combine results from multiple sites in a federated study, existing methods for conducting federated studies can potentially have a problem with consistency across the study sites due to general assumptions that are made to correlate the data. The present disclosure provides a computer-implemented system that enables federated studies to be performed over a set of study sites without having to make general assumptions.

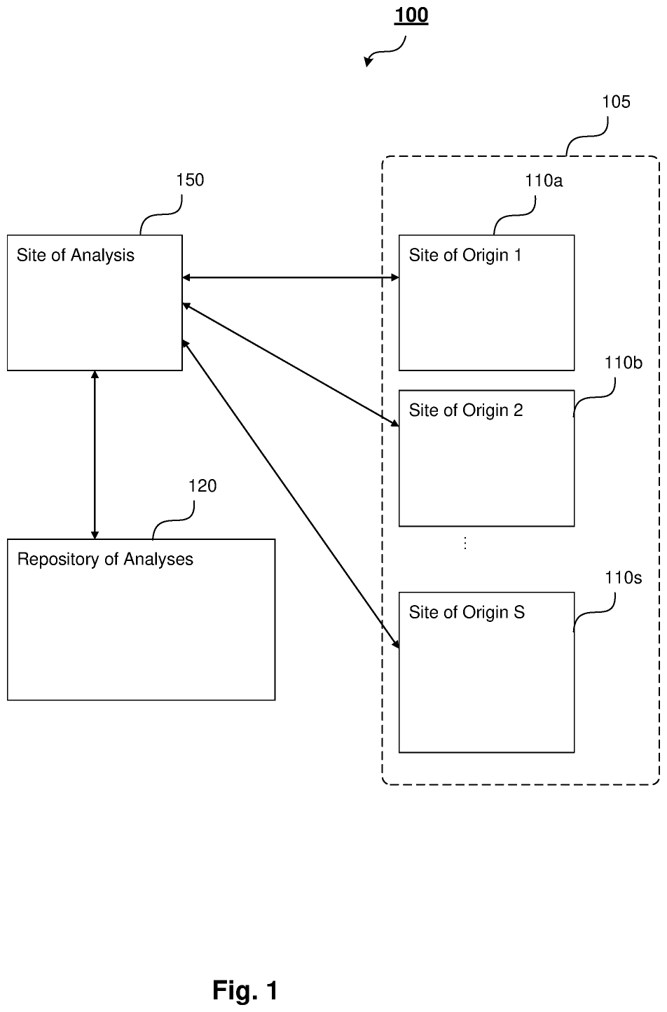

FIG. 1 is a schematic block diagram representation of a computer-implemented system for federated studies 100 in accordance with an embodiment of the present disclosure. The system 100 includes a set 105 of Sites of Origin 110a . . . 110s. Each Site of Origin 110a . . . 110s is a location at which data is acquired in relation to a study and includes an associated data computing device coupled to a communications network to enable communication between each respective Site of Origin 110a . . . 110s to a Site of Analysis 150. The communications network may comprise one or more wired communications links, wireless communications links, or any combination thereof. In particular, the communications network may include a local area network (LAN), a wide area network (WAN), a telecommunications network, or any combination thereof. A telecommunications network may include, but is not limited to, a telephony network, such as a Public Switch Telephony Network (PSTN) or a cellular mobile telephony network, the Internet, or any combination thereof.

The Site of Analysis 150 is a central location for analysing data derived from studies conducted at one or more of the Sites of Origin 110a . . . . 110s. The Site of Analysis 150 is coupled to a Repository of Analyses 120, which is a database that stores a set of different analyses that can be performed and one form of analysis is selected based on the study being performed. Each analysis is a set of computer instructions that specifies how to perform a particular data analysis and each study to be performed is associated with one of the analyses. These computer instructions for performing data analyses can be represented in a computer program, source code, or other electronic document or code that a computer can interpret into a series of operations that result in one or more meaningful Aggregate Values when executed in relation to a set of study data.

The Site of Analysis 150 can obtain data analysis instructions for a given data set in a number of ways. In some embodiments, the Repository of Analyses 120 maintains a link between the Site of Analysis 150 and the Sites of Origin 110a . . . 110s. In alternative embodiments, the instructions for the Site of Analysis 150 include a reference to the instructions to the Sites of Origin 110a . . . 110s.

In further embodiments, the system 100 includes an analysis administrator (not shown) that coordinates analysis instructions among the Sites of Origin 110a . . . 110s and the Site of Analysis 150. The analysis administrator may be implemented using a programmed computing device or a human operator or a combination thereof.

In yet further embodiments, other synchronisation methods, such as Byzantine Agreement, are utilised to ensure that the Site of Analysis 150 utilises a correct set of analysis instructions from the Repository of Analyses 120.

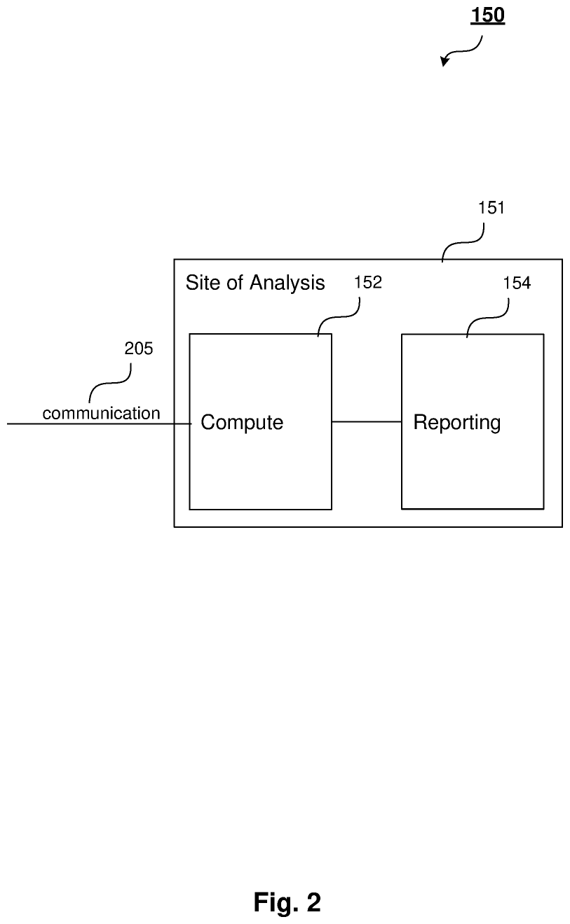

FIG. 2 is an expanded view of functional modules of one embodiment of the Site of Analysis 150. The Site of Analysis 150 includes a computer-implemented data analysis device 151 that is configured to:
Communicate with each database of site aggregate values at each Site of Origin
Interpret the instructions represented in an analysis from the Repository of Analyses into an algorithm
Execute an algorithm interpreted from an analysis from the Repository of Analyses
Report study results

In the context of FIG. 2, the data analysis device 151 receives communication 205 from one or more of the Sites of Origin 110a . . . 110s or the Repository of Analyses 120. Communications between the data analysis device 151 and the Repository of Analyses 120 may be directed to bringing a set of analysis instructions from the Repository of Analyses 120 to the data analysis device 151. Such analysis instructions may then be passed from the data analysis device 151 to one or more of the Sites of Origin 110a . . . 110s.

In some embodiments, the communication 205 relates to a link provided by, or retrieved from, the Repository of Analyses 120 and sent to the data analysis device 151 for forwarding to one or more of the Sites of Origin 110a . . . 110s, such that the respective Site of Origin 110a . . . 110s can utilise the link to retrieve an appropriate set of analysis instructions directly from the Repository of Analyses 120.

The communication 205 may also relate to exchange of information between the data analysis device 151 and one or more of the Sites of Origin 110a . . . 110s, wherein the information may include, for example, requests for aggregated data or the transmission of aggregated data relating to a study.

The received communication 205 is processed by a compute module 152 that interprets instructions received from the Repository of Analyses and applies those instructions to analyse data received from one or more Sites of Origin 110a . . . 110s. The analysed data is then presented to a reporting module 154 that reports study results.

Depending on the implementation, the reporting module 154 reports study results via a user interface (such as a dashboard), by printing results, by emailing results, displaying results to a website, by generating an electronic document, or any combination thereof. The system is configurable to select a set of users to whom study results are sent or who are authorised to access study results. In some embodiments, the level of authorisation allocated to a user determines the level of granularity of the study results that is available to that user. For example, high level users may have access to all levels of study results, whereas low level users may only have access to a macro level of the study results or to a subset of the study results.

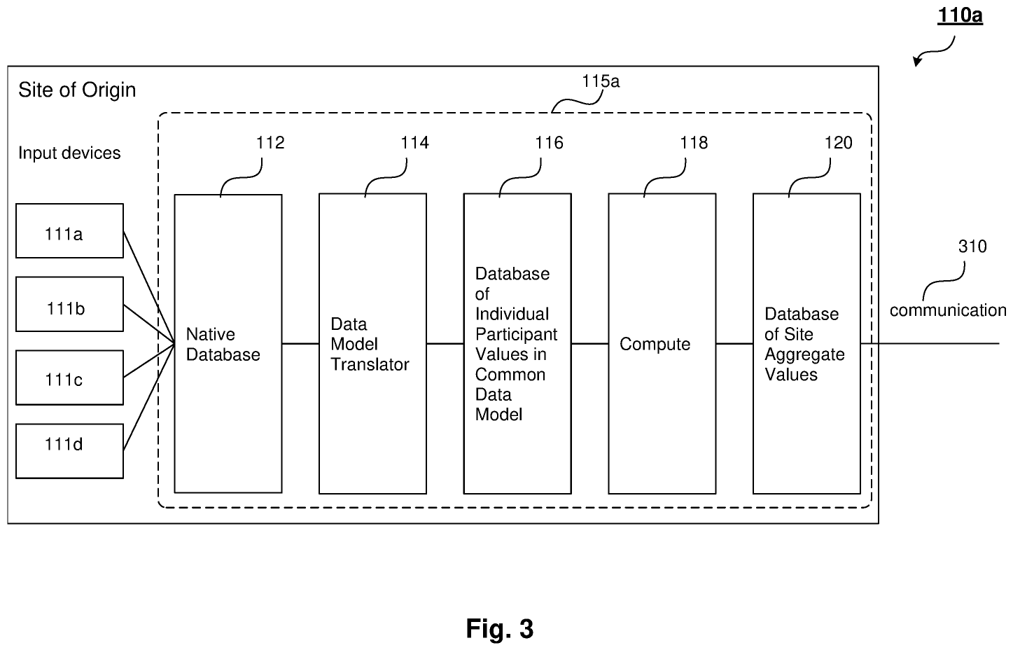

FIG. 3 is a schematic block diagram representation of the Site of Origin 110a of FIG. 1 and is representative of any one of the Sites of Origin 110a . . . 110s. The Site of Origin 110a includes a computer-implemented study data device 115a that is coupled to a set of input devices 111a . . . 111d for acquiring study data in relation to one or more subjects. The input devices 111a . . . 111d may be implemented utilising any suitable device that can be used to digitally capture data that may be used in a federated study. The input devices 111a . . . 111d may include, for example, but are not limited to, electronic health records systems, patient administration systems, computerised provider order entry systems, survey systems, wearable devices, laboratory sensors, imaging machines, electronic thermometers, and the like. Wearable devices may include, for example, but are not limited to, heart rate monitors, blood pressure monitors, electrocardiogram (ECG) monitors, electroencephalograph (EEG) headsets, pulse oximeters, and the like.

For federated studies pertaining to healthcare, the input devices may include anything that generates or captures data in a healthcare setting and such devices are not limited to capturing clinical data, but may equally include devices that capture, acquire, store, or transmit data relating to non-clinical data, such as insurance details, bank details, weather data pertaining to a site of origin (e.g., temperature, barometric pressure, humidity), geographical data pertaining to a site of origin (e.g., altitude), and the like.

Depending on the nature of the input devices 111a . . . 111d, the respective input devices may require manual input from users, may be coupled to the computer-implemented study data device 115a, or may utilise a combination of manual and automated data entry and/or transfer. For example, EEG or ECG monitors may be configured to transmit data directly to the computer-implemented study data device 115a as data is acquired from a patient, whereas patient administration systems and electronic medical records are likely to require at least some manual input of data before transmission to the computer-implemented study data device 115a.

The study data device 115a includes a Native Database 112 that stores Individual Participant Values acquired from one or more of the input devices 111a . . . 111d associated with the respective site of origin, which in the example of FIG. 3 is Site of Origin 110a. In the example of FIG. 3, the Native Database 112 is integral with the study data device 115a. In alternative embodiments, the Native Database 112 may be integral with the respective site of origin, co-located with the site of origin, or remotely located and coupled to the respective site of origin, such as may occur via a cloud-based platform. The Individual Participant Values for each site of origin may be stored in any appropriate data model, depending on the implementation. Further, Individual Participant Values for different studies conducted at a single site of origin may be stored in the same or different data models.

A single study may involve one or more different data sets. Each data set can be associated with a different data model. Alternatively, one or more data sets may share a data model. Relevantly, a translator can be provided to translate different data sets into a common data model. Returning to FIG. 3, Individual Participant Values are captured from one or more of the input devices 111a . . . 111b and then stored in the Native Database 112. A translator 114 translates Individual Participant Values from the data model(s) used in the Native Database(s) into a Common Data Model, wherein the Common Data Model is a predefined data model that is common across all Sites of Origin 110a . . . 110s and the Site of Analysis 150. The translation values are then stored, in the format of the Common Data Model, in a Database of Individual Participant Values in a Common Data Model 116.

An origin compute module 118 interprets instructions represented in an analysis from the Repository of Analyses into an algorithm. The origin compute module 118 then executes the interpreted algorithm in relation to the Database of Individual Participant Values in a Common Data Model 116 and writes the output of the executed algorithm into a Database of Site Aggregate Value 119. The Database of Site Aggregate Values is in communication, via a communications network, with the data analysis device 151 of the Site of Analysis 150.

During the course of a study, Aggregate Values may be exchanged back and forth between one or more of the Sites of Origin 110a . . . s and the Site of Analysis 150 multiple times, as the Aggregate Values are computed. The nature of what aggregate values are sent and when those aggregate values are sent is defined by, and coordinated by, sets of send and retrieve instructions within the analysis instructions. Such analysis instructions may be contained within a predefined analysis profile that forms part of the analysis instructions defined for a particular study.

### Common Data Model

Each site of origin is assumed to have the Individual Participant Values needed to calculate the site aggregates according to an algorithm, referred to herein as Analysis_{O}. However, Individual Participant Values can be represented in a number of ways (Data Models), including in a manner that is unique to that particular site.

The site of analysis needs to agree with each site of origin on a common data model to be used in communications. The common data model can be a pre-defined common data model or alternatively a common data model specific to the study may be utilised.

The Data Model Translation system at each Site of Origin, sometimes known as an Extract, Transform and Load (ETL), is configured specifically for the site of origin and its Native Databases. However, the output of the Data Model Translator at each site of origin represents the same Individual Participant Values in the Common Data Model.

### Preconditions

One site of analysis is identified. For convenience, the site of analysis is referred to herein as Site_{A}.
One or more sites of origin are identified. Sites of origin are uniquely named such that the site of analysis can address each site of origin. For convenience, herein, sites of origin are named Site₁, Site₂, etc., where the last site is Site_{S}, and to every site of origin generically as Siteᵢ.
The Common Data Model is used by both Site_{A} and Siteᵢ.
One or more Data Model Translators are set up in each Siteᵢ.
It is possible that one site of origin coincides with Site_{A}, but this is not assumed to be the case.
Network permissions and access are configured such that:
   the site of analysis can access Aggregate Data on each site of origin,
   the site of analysis can access and download analyses from the Repository of Analyses
   Each site of origin can either:
      access and download analyses from the Repository of Analyses as directed by the site of analysis, or
      be sent analyses from the site of analysis
The Repository of Analyses has an analysis that instructs a site of origin on how to calculate one or more Site Aggregates. For convenience, this analysis will be referred to herein as Analysis_{O}, regardless of the number of Aggregate Values it calculates.
Analysis_{O} can use Individual Participant Values, Site Aggregates, Study Aggregates, or any combination thereof.
The Repository of Analyses has at least one analysis that instructs a site of analysis on how to calculate one or more Study Aggregates. For convenience, we refer to this analysis herein as Analysis_{A}.
Analysis_{A} is marked in the Repository of Analyses as being linked to Analysis_{O}. That is, Analysis_{O} can be used by more than one Analysis_{A}. Each unique study has its own Analysis_{A} coupled with one or more Analysis_{O}. When a study is repeated (for example, at a later time, or at another set of sites), the same set of analyses can be used again.
Analysis_{A} specifies a set of Analysis_{O} on which that Analysis_{A} relies, but any given Analysis_{O} is not restricted to be used by only one Analysis_{A}. For example, Analysis_{O} may relate to “count the number of asthmatic patients” and that Analysis_{O} can be used by any study of asthmatic patients.
Analysis_{A} doesn't use Site Aggregates that are not calculated by an Analysis_{O}.
Analysis_{A} doesn't use Individual Participant Values.

### Instigation

The calculation of the Task can be instigated in one of three ways:
1. The study is initiated by Site_{A} at a scheduled time, or as a result of user input:
   a. Site_{A} notifies each Siteᵢ of the intention to synchronise the study start time immediately,
   b. Each Siteᵢ acknowledges the request with a message to Site_{A}.
      i. In some implementations, Siteᵢ can respond with a request to reschedule and suggest an alternative time.
      ii. In such cases, Site_{A} will send a cancellation to each Siteᵢ and repeat this Step 1 at a later time.
      iii. 1.b.i and 1.b.ii can be repeated until all Siteᵢ are ready.
2. The study is initiated at a set time
   a. Each Siteᵢ notifies Site_{A} of its readiness to conduct the study
3. The study is initiated when
   a. each Siteᵢ notifies Site_{A} that a condition is met which makes it ready to conduct the study,
   b. Site_{A} waits until it receives such messages from each Siteᵢ
   c. Site_{A} initiates the study immediately as per Step 1 above

Regardless of the instigation method used, at the end of the Instigation, all sites have the information they need to conduct the study and are ready to continue.

### Synchronizing Analyses

1. Site_{A} downloads the latest Analysis_{A}, that is, a computational representation of the procedure used to calculate the Study Aggregate(s) from Site Aggregates.
2. Site_{A} either:
   a. Instructs each Siteᵢ to download the same Analysis_{O} by
      i. identifying Analysis_{O} associated with Analysis_{A},
      ii. sending the identity of Analysis_{O} to each Siteᵢ so that
      iii. each Siteᵢ downloads the same analysis from the Repository of Analyses
   b. Or, downloads the Analysis_{O} associated with Analysis_{A} and uploads it to each Siteᵢ

At the end of this stage, Site_{A} has a copy of Analysis_{A} and each Siteᵢ has an identical copy of the associated Analysis_{O}.

### Analysis Structure

Each Analysis is a representation of one or more algorithms that perform part of the study.
Analysis_{O} uses Individual Participant Values, Site Aggregates and Study Aggregates to calculate Site Aggregates.
Analysis_{A} uses Site Aggregates to calculate the Study Aggregate.
Analysis_{O} can include implicit or explicit instructions to temporarily pause execution of the algorithm until required Study Aggregate(s) are present.
Analysis_{A} can include instructions to temporarily pause execution of the algorithm until required Site Aggregates are present.
Analysis_{A} can include implicit or explicit instructions to send Study Aggregates to each Siteᵢ.
The output of Analysis_{O} are all Site Aggregates required to complete Analysis_{A}.
The output of Analysis_{A} is all Study Aggregates, for example in the form of:
   a text report,
   a report in a format that can be sent to an automated reporting system such as required by a public health or another authority, and/or
   values in a form interpretable by another system such as a dashboard or a data visualisation system.

### Computer Implementation

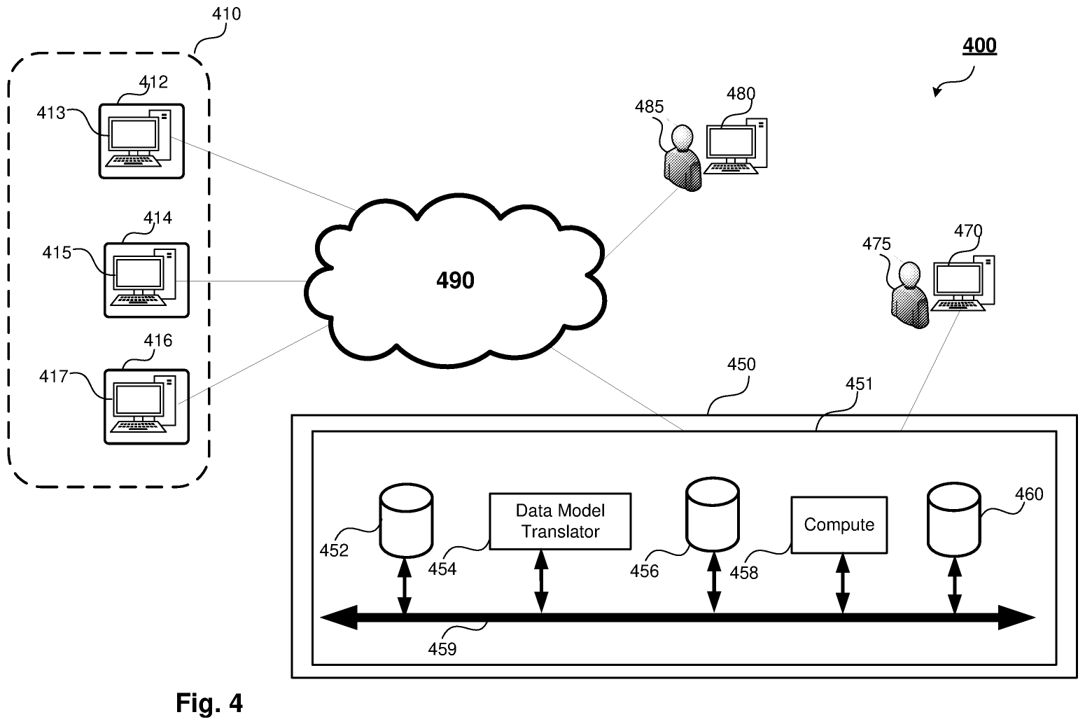

FIG. 4 is a schematic block diagram representation of a federated studies system 400 on which one or more embodiments of the present disclosure may be practised. The system 400 includes a set 410 of sites of origin that includes Site₁ 412, Site₂ 414, and Site₃ 416. The Sites of Origin_{1 . . . 3} 412, 414, 416 may be co-located or located in separate locations, or a combination thereof.

Each Site of Origin 412, 414, 416 is associated with at least one respective data computing device 413, 415, 417 that is coupled to a communications network 490. The data computing devices 413, 415, 417 transmit aggregated data via the communications network 490. The communications network 490 may comprise one or more wired communications links, wireless communications links, or any combination thereof. In particular, the communications network 490 may include a local area network (LAN), a wide area network (WAN), a telecommunications network, or any combination thereof. A telecommunications network may include, but is not limited to, a telephony network, such as a Public Switch Telephony Network (PSTN) or a cellular mobile telephony network, the Internet, or any combination thereof.

The system 400 also includes a Site of Analysis 450. The Site of Analysis 450 includes a Study Data Device 451 that includes: a Native Database 452, a Data Model Translator 454, a Database of Individual Participant Values in Common Data Model 456, a Compute module 458, and a Database of Site Aggregate Values 460, each of which is coupled to a communications bus 459 that enables exchange of information across each of the components of the Site of Analysis 450.

The Data Model Translator 454 is utilised in scenarios in which the Site of Analysis 450 needs to translate received data into a common data model format. In scenarios in which the Sites of Origin only transmit aggregated data in accordance with a predefined common data model, then the Data Model Translator 454 is not necessary.

The Study Data Device 451 is coupled to the communications network 490 to enable communication between the Site of Analysis 450 and each of the Sites of Origin 412, 414, 416. In particular, the Sites of Origin 412, 414, 416 utilise the respective data computing devices 413, 415, 417 to transmit aggregated data sets pertaining to one or more studies, via the communications network 490, to the Study Data Device 451.

In the example of FIG. 4, the system 400 further optionally includes a first observational computing device 470 and an associated first observer 475. The first observer 475 is able to access the first observational computing device 470 to access a user interface provided by the Site of Analysis 450 in order to view data associated with one or more federated studies. The first observational computing device 470 may be integral with the Study Data Device 451 or coupled to the Study Data Device via one or more wired and/or wireless communications links.

In the example of FIG. 4, the system 400 further optionally includes a second, remote observational computing device 480 and an associated second observer 485. The remote observational computing device 480 is coupled to the Study Data Device 451 via one or more wired and/or wireless communications links, including the communications network 490. The second observer 485 is able to access the remote observational computing device 480 to access a user interface provided by the Site of Analysis 450 in order to view data associated with one or more federated studies.

The functionality of the federated studies system 400 will now be described in relation to an example study pertaining to the body weight of patients with Type 2 diabetes.

Example: Mean (Ŷ) and Standard Deviation (σ) of Body Weights of Patients with a Type 2 Diabetes Mellitus Diagnosis from 2010 in 3 Sites

### Sites of Origins' Input Devices and Native Databases

Sites of Origin (Site₁, Site₂ and Site₃) are healthcare sites.
Each Site of Origin has an associated electronic health record system.
Site₁, has electronic scales that automatically record the body weight in kilograms (kg) in the patient's electronic health record.
In Site₂, a doctor weighs patients and inputs the body weight in kg in the patient's electronic health record.
In Site₃, a nurse weighs patients and inputs the patient's body weight in pounds (lb) in the patient's electronic health record.
In each Site of Origin, a doctor enters a Type 2 Diabetes Mellitus diagnosis in a diagnosed patient's electronic health record.

### Common Data Model

In this example, the common data model for the study includes a Conditions Table and an Observations Table. In different embodiments relating to different studies, the common data model may include one or more tables or other suitable data formats. In this example, the information in the Conditions Table and the Observations Table is populated from the Native Database at the respective Site of Origin.

Some information in the Conditions Table and the Observation Table of the common data model used in this example utilises codes from the SNOMED-CT (snomed.org) clinical terminology, which provides a translation of these codes to a standardised, unambiguous textual description of the condition, observation, etc. In this example, the text descriptions of the relevant codes are not included in the common data model, but are given in the body of the text for convenience.

Type 2 Diabetes Mellitus diagnoses are recorded in a “Conditions” table, with the patient's unique identification number (UID), the date and time of the measurement, and the SNOMED-CT code 44054006 (“Type 2 Diabetes Mellitus”) as illustrated in Table 1 below:

TABLE 1
Conditions

| UID | Date + time | condition code |
|---|---|---|
| 1 | Apr. 21, 2010 14:35 | 44054006 |

Body weight is recorded in an “Observations” table with the patient's UID, with the SNOMED code 27113001 (“Body Weight”) and the corresponding value in kg and the unit as a SNOMED code 258683005 (“kg”) as illustrated in Table 2 below.

TABLE 2
Observations

| | | observation | observation | |
|---|---|---|---|---|
| UID | Date + Time | code | value | unit code |
| 1 | Apr. 21, 2010 | 27113001 | 98.3 | 258683005 |
| | 14:30 | | | |

### Sites of Origins' Data Model Translators

Each Site of Origin has its own translator, such as the Data Model Translator 114 of FIG. 1, to translate data from its own electronic health record system to the common data model. In Site₃, the translator includes converting body weights from pounds (Ib) to kilograms (kg).

### Example: Calculating Mean and Standard Deviation in Body Weight of Participants in a Federated Study

### Formulae to Calculate Mean Date Time (Ŷ) and Standard Deviation (σ)

$$\begin{matrix}\hat{Y} = \frac{1}{N} {\sum }_{j = 1}^{N} & {Y}_{j}\end{matrix} \qquad Eqn ( 1 )$$

$$σ = \sqrt{\begin{matrix}\frac{1}{N} {\sum }_{j = 1}^{N} & {( {Y}_{j} - \hat{Y} )}^{2}\end{matrix}} \qquad Eqn ( 2 )$$

### Analysis_{A} Pseudocode

1. Request and wait for number of included participants (n₁, n₂ and n₃) from each corresponding Site of Origin
2. Calculate the Study Aggregate “number of included patients in study” N as

$$N = \begin{matrix}{\sum }_{i = 1}^{3} & {n}_{i}\end{matrix}$$

3. Request and wait for Site Aggregate “site mean body weight” (Ŷ₁, Ŷ₂ and Ŷ₃) from each corresponding Site of Origin
4. Calculate Study Aggregate “study mean body weight” Ŷ as

$$\hat{Y} = \frac{1}{N} {\sum }_{i = 1}^{3} \qquad {\hat{Y}}_{i} \cdot {n}_{i}$$

5. Request and wait for Site Aggregate “sum of square differences” (\hat{Z}₁, \hat{Z}₂ and \hat{Z}₃) from each corresponding Site of Origin
6. Calculate Study Aggregate “standard deviation” σ from Site Aggregates “sum of square differences” (\hat{Z}₁, \hat{Z}₂, \hat{Z}₃) and Study Aggregate “number of included patients in study” N as

$$σ = \sqrt{\begin{matrix}\frac{1}{N} {\sum }_{i = 1}^{3} & {\hat{Z}}_{i} \cdot {n}_{i}\end{matrix}}$$

7. Report Study Aggregate “study mean body weights” Ŷ and “standard deviation” σ

### Analysis_{O} Pseudocode

1. Set site number as i
2. Count number of unique included patients with the condition code 44054006 with condition date and time between Jan. 1, 2010 12:00 am and Dec. 31, 2010, 11:59 pm as nᵢ
3. Calculate the “site mean body weight” Ŷᵢ from the Individual Participant Value body weight (Y) for each included patient j and the number of included patients at the site n as

$${\hat{Y}}_{i} = \begin{matrix}\frac{1}{n} {\sum }_{j = 1}^{n} & {Y}_{j}\end{matrix}$$

4. Request and wait for Study Aggregate “study mean body weight” Ŷ from Site_{A}
5. Calculate Site Aggregate “sum of square differences” \hat{Z}ᵢ from the body weight for each included patient j and the Study Aggregate “study mean body weight” Ŷ as

$${\hat{Z}}_{i} = \begin{matrix}{\sum }_{j = 1}^{n} & {( {Y}_{j} - \hat{Y} )}^{2}\end{matrix}$$

The federated studies system of the present disclosure may be practised using one or more computing devices, such as a general purpose computer, computer server, distributed cloud-based architecture, a combination thereof, or any similar architecture that is programmed to perform one or more of the functions shown and described in relation to FIGS. 1 to 4, thus giving rise to a new and improved computing device. In particular, the study data device 115a and data analysis device 451 are advances in computer technology implemented utilising one or more computing devices.

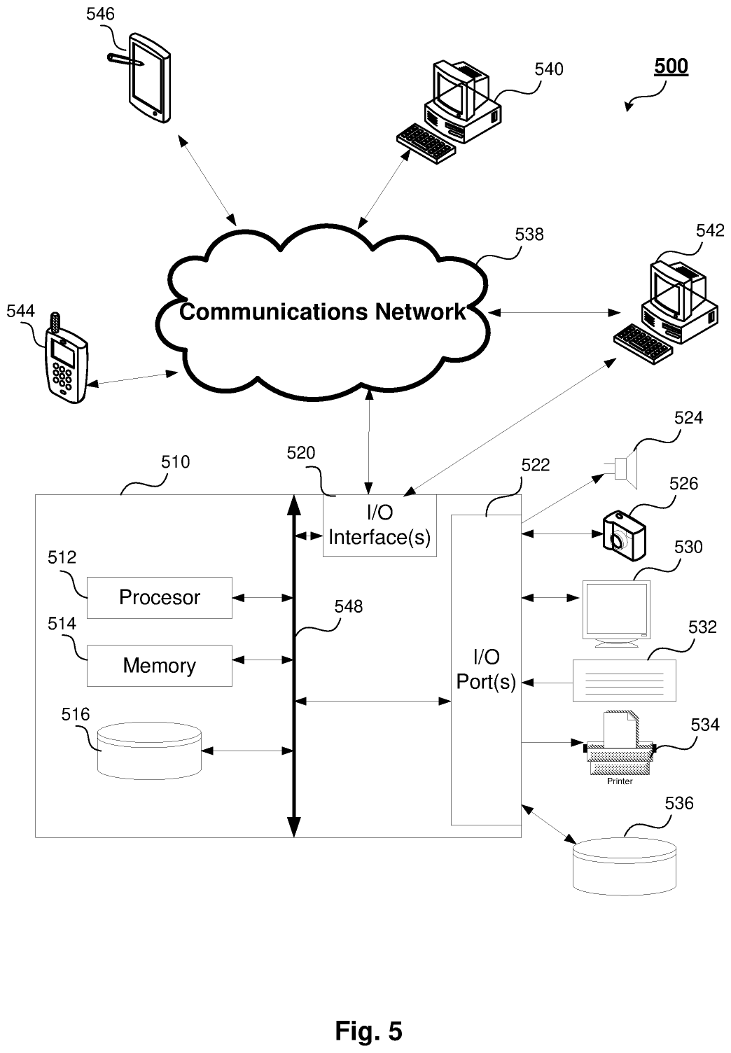

FIG. 5 is a schematic block diagram of a system 500 that includes a general purpose computer 510. The general purpose computer 510 includes a plurality of components, including: a processor 512, a memory 514, a storage medium 516, input/output (I/O) interfaces 520, and input/output (I/O) ports 522. Components of the general purpose computer 510 generally communicate using one or more buses 548.

The memory 514 may be implemented using Random Access Memory (RAM), Read Only Memory (ROM), or a combination thereof. The storage medium 516 may be implemented as one or more of a hard disk drive, a solid state “flash” drive, an optical disk drive, or other storage means. The storage medium 516 may be utilised to store one or more computer programs, including an operating system, software applications, and data. In one mode of operation, instructions from one or more computer programs stored in the storage medium 516 are loaded into the memory 514 via the bus 548. Instructions loaded into the memory 514 are then made available via the bus 548 or other means for execution by the processor 512 to implement a mode of operation in accordance with the executed instructions.

One or more peripheral devices may be coupled to the general purpose computer 510 via the I/O ports 522. In the example of FIG. 5, the general purpose computer 510 is coupled to each of a speaker 524, a camera 526, a display device 530, an input device 532, a printer 534, and an external storage medium 536. The speaker 524 may be implemented using one or more speakers, such as in a stereo or surround sound system. In the example in which the general purpose computer 510 is utilised to implement one or more of the functions of a federated studies system, such as a study data device, one or more peripheral devices may relate to input devices 111a . . . d of FIG. 3 connected to the I/O ports 522 either wirelessly or by wired connection.

The camera 526 may be a webcam, or other still or video digital camera, and may download and upload information to and from the general purpose computer 510 via the I/O ports 522, dependent upon the particular implementation. For example, images recorded by the camera 526 may be uploaded to the storage medium 516 of the general purpose computer 510. Similarly, images stored on the storage medium 516 may be downloaded to a memory or storage medium of the camera 526. The camera 526 may include a lens system, a sensor unit, and a recording medium.

The display device 530 may be a computer monitor, such as a cathode ray tube screen, plasma screen, or liquid crystal display (LCD) screen. The display 530 may receive information from the computer 510 in a conventional manner, wherein the information is presented on the display device 530 for viewing by a user. The display device 530 may optionally be implemented using a touch screen to enable a user to provide input to the general purpose computer 510. The touch screen may be, for example, a capacitive touch screen, a resistive touchscreen, a surface acoustic wave touchscreen, or the like.

The input device 532 may be a keyboard, a mouse, a stylus, drawing tablet, or any combination thereof, for receiving input from a user. The external storage medium 536 may include an external hard disk drive (HDD), an optical drive, a floppy disk drive, a flash drive, solid state drive (SSD), or any combination thereof and may be implemented as a single instance or multiple instances of any one or more of those devices. For example, the external storage medium 536 may be implemented as an array of hard disk drives.

The I/O interfaces 520 facilitate the exchange of information between the general purpose computing device 510 and other computing devices. The I/O interfaces may be implemented using an internal or external modem, an Ethernet connection, or the like, to enable coupling to a transmission medium. In the example of FIG. 5, the I/O interfaces 522 are coupled to a communications network 538 and directly to a computing device 542. The computing device 542 is shown as a personal computer, but may equally be practised using a smartphone, laptop, or a tablet device. Direct communication between the general purpose computer 510 and the computing device 542 may be implemented using a wireless or wired transmission link.

The communications network 538 may be implemented using one or more wired or wireless transmission links and may include, for example, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a mobile telephone cellular network, a short message service (SMS) network, or any combination thereof. The general purpose computer 510 is able to communicate via the communications network 538 to other computing devices connected to the communications network 538, such as the mobile telephone handset 544, the touchscreen smartphone 546, the personal computer 540, and the computing device 542.

One or more instances of the general purpose computer 510 may be utilised to implement one or more functions of the study data device 115a of FIG. 3 to implement a Site of Origin of a federated studies system in accordance with the present disclosure. In such an embodiment, the memory 514 and storage 516 are utilised to store data relating to patient data, analysis data, and the like. Software for implementing the federated studies system is stored in one or both of the memory 514 and storage 516 for execution on the processor 512. The software includes computer program code for implementing method steps in accordance with the functional modules described herein.

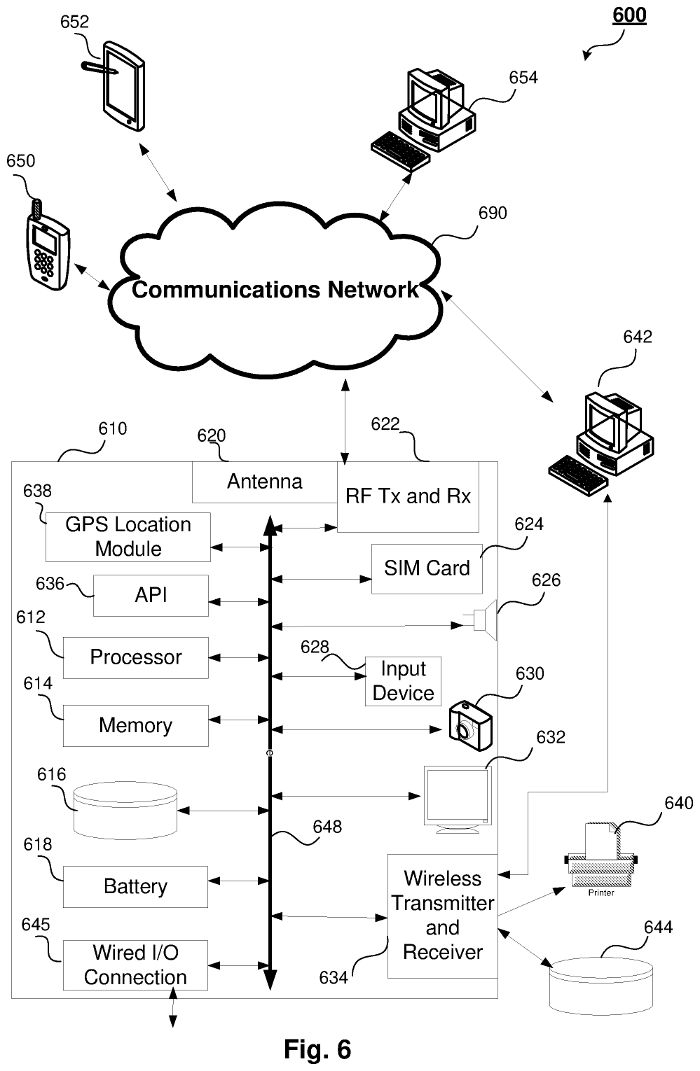

FIG. 6 is a schematic block diagram of a system 600 on which one or more aspects of a federated method and system of the present disclosure may be practised. The system 600 includes a portable computing device in the form of a smartphone 610, which may be used by a registered user of the federated studies system in FIG. 1. The smartphone 610 includes a plurality of components, including: a processor 612, a memory 614, a storage medium 616, a battery 618, an antenna 620, a radio frequency (RF) transmitter and receiver 622, a subscriber identity module (SIM) card 624, a speaker 626, an input device 628, a camera 630, a display 632, and a wireless transmitter and receiver 634. Components of the smartphone 610 generally communicate using one or more bus connections 648 or other connections therebetween. The smartphone 610 also includes a wired connection 645 for coupling to a power outlet to recharge the battery 618 or for connection to a computing device, such as the general purpose computer 510 of FIG. 5. The wired connection 645 may include one or more connectors and may be adapted to enable uploading and downloading of content from and to the memory 614 and SIM card 624.

The smartphone 610 may include many other functional components, such as an audio digital-to-analogue and analogue-to-digital converter and an amplifier, but those components are omitted for the purpose of clarity. However, such components would be readily known and understood by a person skilled in the relevant art.

The memory 614 may include Random Access Memory (RAM), Read Only Memory (ROM), or a combination thereof. The storage medium 616 may be implemented as one or more of a solid state “flash” drive, a removable storage medium, such as a Secure Digital (SD) or microSD card, or other storage means. The storage medium 616 may be utilised to store one or more computer programs, including an operating system, software applications, and data. In one mode of operation, instructions from one or more computer programs stored in the storage medium 616 are loaded into the memory 614 via the bus 648. Instructions loaded into the memory 614 are then made available via the bus 648 or other means for execution by the processor 612 to implement a mode of operation in accordance with the executed instructions.

The smartphone 610 also includes an application programming interface (API) module 636, which enables programmers to write software applications to execute on the processor 612. Such applications include a plurality of instructions that may be pre-installed in the memory 614 or downloaded to the memory 614 from an external source, via the RF transmitter and receiver 622 operating in association with the antenna 620 or via the wired connection 645.

The smartphone 610 further includes a Global Positioning System (GPS) location module 638. The GPS location module 638 is used to determine a geographical position of the smartphone 610, based on GPS satellites, cellular telephone tower triangulation, or a combination thereof. The determined geographical position may then be made available to one or more programs or applications running on the processor 612.

The wireless transmitter and receiver 634 may be utilised to communicate wirelessly with external peripheral devices via Bluetooth, infrared, or other wireless protocol. In the example of FIG. 6, the smartphone 610 is coupled to each of a printer 640, an external storage medium 644, and a computing device 642. The computing device 642 may be implemented, for example, using the general purpose computer 510 of FIG. 5.

The camera 626 may include one or more still or video digital cameras adapted to capture and record to the memory 614 or the SIM card 624 still images or video images, or a combination thereof. The camera 626 may include a lens system, a sensor unit, and a recording medium. A user of the smartphone 610 may upload the recorded images to another computer device or peripheral device using the wireless transmitter and receiver 634, the RF transmitter and receiver 622, or the wired connection 645.

In one example, the display device 632 is implemented using a liquid crystal display (LCD) screen. The display 632 is used to display content to a user of the smartphone 610. The display 632 may optionally be implemented using a touch screen, such as a capacitive touch screen or resistive touchscreen, to enable a user to provide input to the smartphone 610.

The input device 628 may be a keyboard, a stylus, or microphone, for example, for receiving input from a user. In the case in which the input device 628 is a keyboard, the keyboard may be implemented as an arrangement of physical keys located on the smartphone 610. Alternatively, the keyboard may be a virtual keyboard displayed on the display device 632.

The SIM card 624 is utilised to store an International Mobile Subscriber Identity (IMSI) and a related key used to identify and authenticate the user on a cellular network to which the user has subscribed. The SIM card 624 is generally a removable card that can be used interchangeably on different smartphone or cellular telephone devices. The SIM card 624 can be used to store contacts associated with the user, including names and telephone numbers. The SIM card 624 can also provide storage for pictures and videos. Alternatively, contacts can be stored on the memory 614.

The RF transmitter and receiver 622, in association with the antenna 620, enable the exchange of information between the smartphone 610 and other computing devices via a communications network 690. In the example of FIG. 6, RF transmitter and receiver 622 enable the smartphone 610 to communicate via the communications network 690 with a cellular telephone handset 650, a smartphone or tablet device 652, a computing device 654 and the computing device 642. The computing devices 654 and 642 are shown as personal computers, but each may be equally be practised using a smartphone, laptop, or a tablet device.

The communications network 690 may be implemented using one or more wired or wireless transmission links and may include, for example, a cellular telephony network, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a cellular (mobile) telephone cellular network, a short message service (SMS) network, or any combination thereof.

When one or more functions of the federated studies system described herein are implemented using the smartphone 610 of FIG. 6, a software application (“app”) executing on the processor 612 may be utilised to implement any one or more of the functions described and shown in relation to FIGS. 1 to 4. In some implementations, the app is a native app executing on the smartphone 600. In alternative implementations, the app is a web-based app displayed in a browser executing on the processor 612, with the smartphone 610 coupled to a remote server, such as the computing device 642, on which the app is executing.

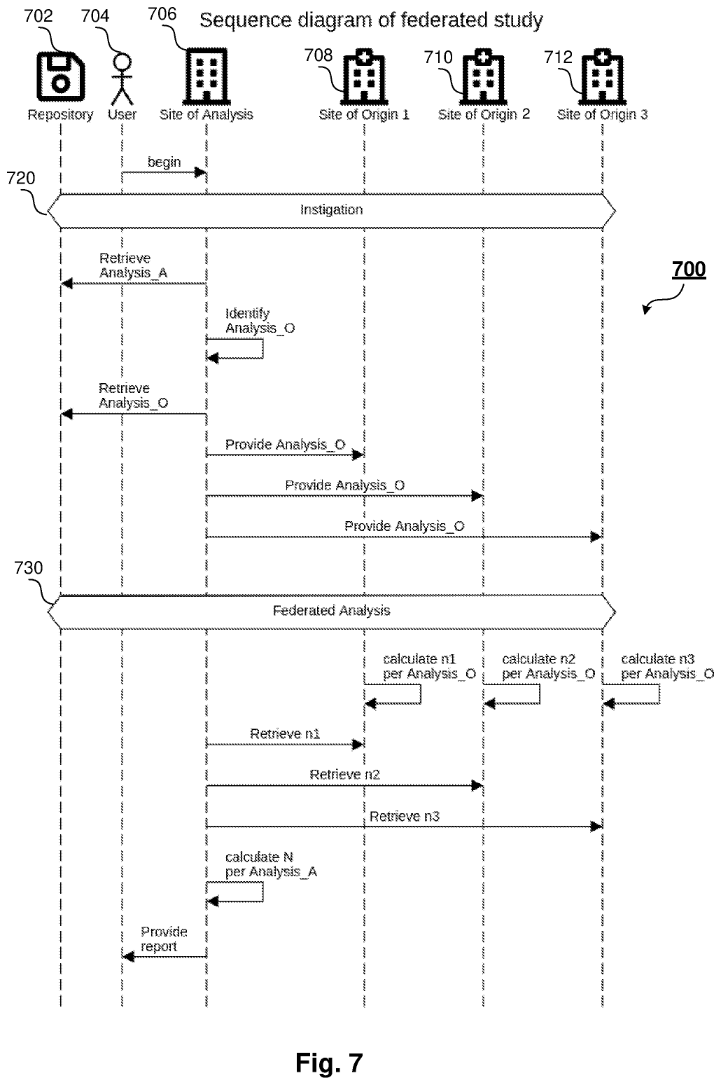

FIG. 7 is a sequence diagram illustrating a practice workflow 700 of a federated study performed by an embodiment of the system of the present disclosure. The workflow 700 relates to a system having a repository 702, a user 704, a site of analysis 706, and a set of three sites of origin, respectively a first site of origin 708, a second site of origin 710, and a third site of origin 712.

At a first step, the user 704 begins a federated study at the site of analysis 706, with instigation of the study commencing with the site of analysis 706 retrieving AnalysisA from the repository 702. As described above, each AnalysisA is associated with a set of one or more AnalysisOs. The site of analysis 706 identifies the AnalysisO associated with the retrieved AnalysisA and then retrieves the identified AnalysisO from the repository 702.

The site of analysis 706 then provides the retrieved AnalysisO to each of the first site of origin 708, the second site of origin 710, and the third site of origin 712. Thus, each site of origin 708, 710, 712 has AnalysisO by which to conduct a study.

Each site of origin 708, 710, 712 then aggregates study data for the respective site of origin. In FIG. 7, this is shown by the first site of origin 708 calculating n1 per AnalysisO, the second site of origin 710 calculating n2 per AnalysisO, and the third site of origin 712 calculating n3 per AnalysisO.

The site of analysis 706 retrieves the calculated n1, n2, and n3 from the respective sites of origin 708, 710, 712 and then calculates N in accordance with AnalysisO. The site of analysis 706 then provides a report to the user 704, based on the calculated N.

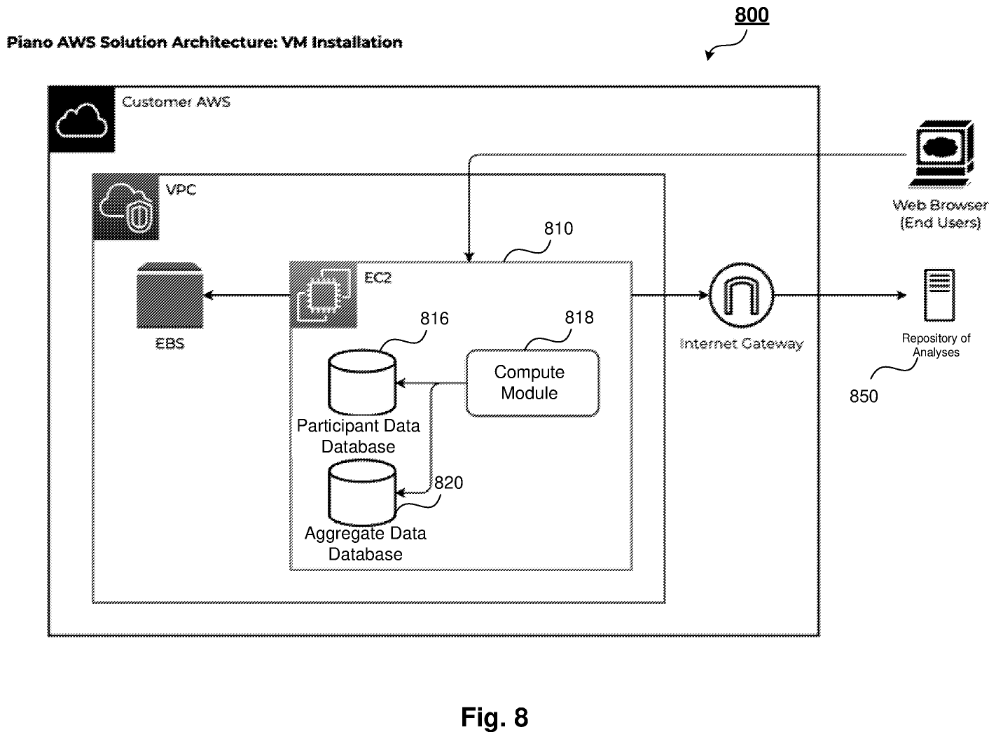

FIG. 8 is schematic block diagram representation of a cloud-based virtual machine architecture for implementing a site of origin 800 in a system of the present disclosure. The example of FIG. 8 utilises the cloud-based computing platform AWS provided by Amazon Web Services, Inc. In particular, the Amazon Virtual Private Cloud (VPC) is used to implement the site of origin, with Amazon Elastic Block Store (EBS) being used as block-level storage to store data. Such data may include, for example, patient data, study data, AnalysisO, or the like.

The example of FIG. 8 also utilises Amazon Elastic Cloud Compute (EC2) to perform as a virtual machine 810 hosting a software application for implementing functionality of the site of origin. The virtual machine 810 includes a compute module 818 corresponding to the compute module 118 of FIG. 3, a participant data database 816 corresponding to the database 116 of FIG. 3, and an aggregate data database 820 corresponding to the aggregate values database 120 of FIG. 3.

The site of origin 800 is accessible, via the Internet, by end users accessing web browsers executing on computing devices. The site of origin 800 is also coupled, via an Internet Gateway, to a database 850 functioning as a repository of analyses.

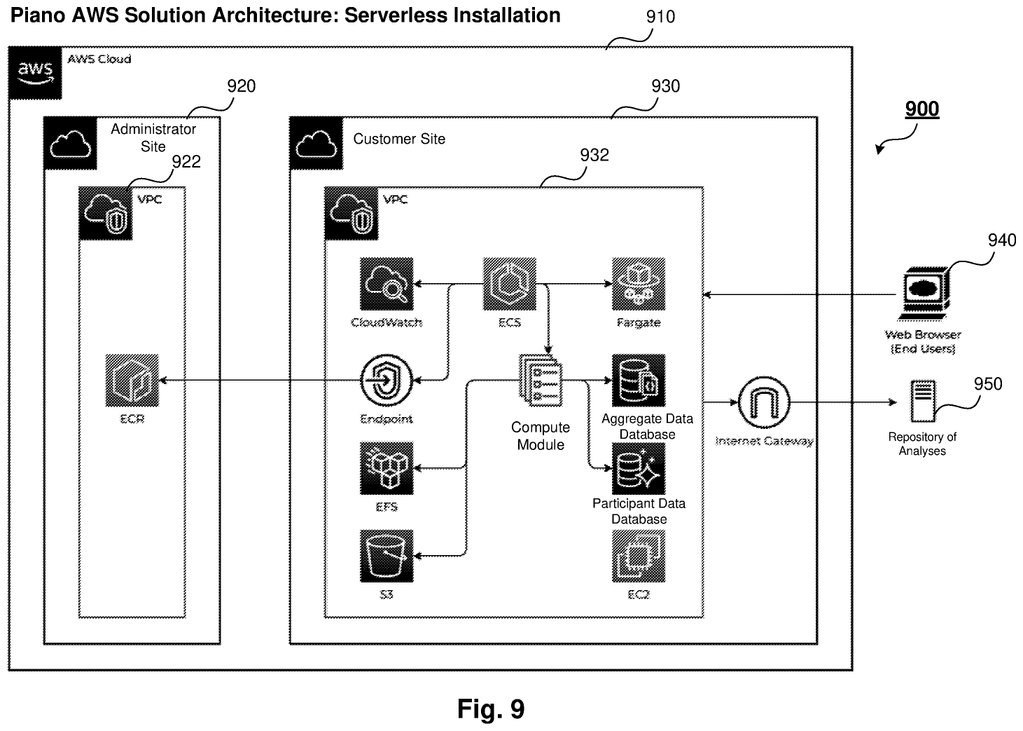

FIG. 9 is a schematic block diagram representation of a Site of Origin system 900 implemented utilising a cloud-based “serverless” computer architecture 910, which in this example is provided by Amazon Web Services. An administrator site 920 is implemented using a virtual machine that includes a software distribution system Elastic Container Registry (ECR) for distributing software to a customer site 930.

The customer site 930 is implemented using a virtual machine 932 that implements the site of origin 110a of FIGS. 1 and 3. The customer site 930 includes a number of functional components, including a compute module, aggregate data database, and a participant data database, as set out in, and described with reference to, FIG. 3. The customer site 930 is coupled, via one or more communications links, to each of an end user computer 940 and a repository of analyses 950.

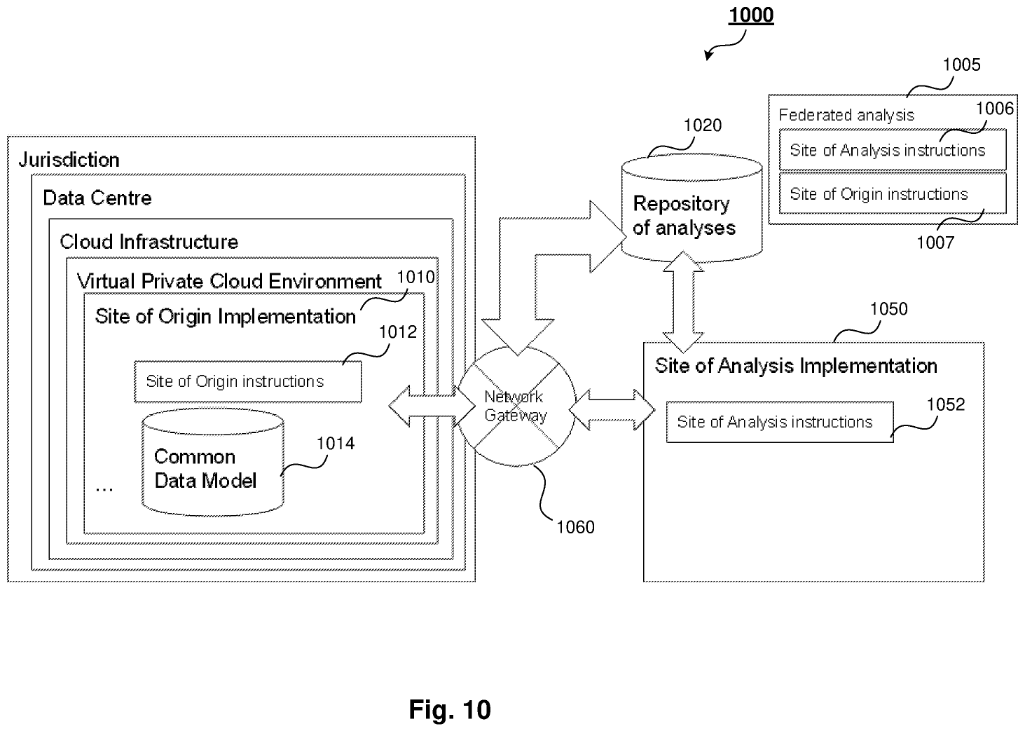

FIG. 10 is a schematic block diagram representation of a federated studies system 1000 implemented utilising a cloud-based computer architecture. The system 1000 includes a federated analysis module 1005 that is a conceptual representation of how a site of analysis instructions set 1006 and site of origin instructions set 1007 might be arranged, so as to appear as a single functional module.

A site of origin 1010 is implemented using a cloud-based system, such as that described with reference to FIG. 8. The site of origin 1010 includes a set of site of origin instructions 1012 and a common data model 1014 defined in relation to a particular federated study. The site of origin 1010 is coupled to a network gateway 1060 that enables exchange of data among the site of origin 1010, a site of analysis 1050, and a repository of analyses 1020. The site of analysis 1050 includes a set of site of analysis instructions 1052.

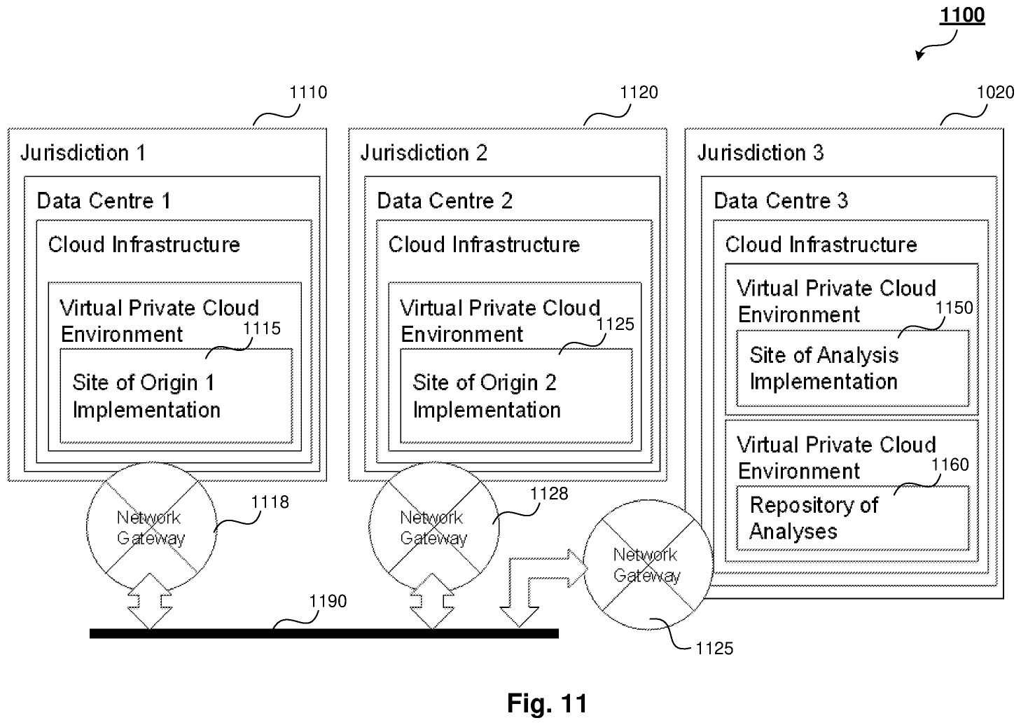

FIG. 11 is a schematic block diagram representation of a federated studies system 1100 implemented utilising a cloud-based computer architecture. The system 1100 in the example of FIG. 11 includes a first site of origin 1115 hosted at a first data centre, a second site of origin 1125 hosted at a second data centre, a site of analysis 1150 hosted at a third data centre, and a repository of analyses 1160, each of which is implemented in this example in a cloud-based computing environment.

The first site of origin 1115 is coupled to a first network gateway 1118, which is coupled to a communications network 1190. The second site of origin 1125 is coupled to a second network gateway 1128, which is also coupled to the communications network 1190. The site of analysis 1150 and repository of analyses 1160 are co-located in a common, third data centre and are coupled, via a third network gateway 1125 to the communications network 1190.

The communications network 1190 may be implemented using one or more wired or wireless communications links, and may include the Internet, that enable transmission of data among the first site of origin 1115, the second site of origin 1125, the site of analysis 1150, and the repository of analyses 1160.

The federated studies system 1100 of FIG. 11 enables two sites of origin 1115, 1125 to collect participant data from separate sites and then transmit that data, according to a predefined common data model, for processing by the site of analysis 1150. Such a federated studies system 1100 provides an improved computing architecture for conducting federated studies.

### Data Model Dialect Unification

The plurality of representations for data across separate sites can produce variability in the “model” or “format” of the data in a data repository, such as a relational database or a data lake. This variability can introduce errors to analysis, retrieval, and update of data in such repositories, especially when multiple repositories are combined either by aggregation (copying content from one or more repositories to another) or federation (combining summary information derived at each repository).

In order to address this problem, several common data standards have been proposed, including FHIR, OMOP and I2B2 in medicine. However, to translate data into such common data models often requires expertise, subjectivity, and judgement to be applied to the translation, still leaving some differences in the model of the translated and standardised data (sometimes called ‘dialects’). These differences across the dialects limit the ability to combine data even from repositories using the same common data model.

There are two main reasons dialects occur: Interpretation and Omission. Interpretation occurs when the instructions of how to model data to a standard are ambiguous or unclear in some other way, leaving room for data transformers at different sites and/or at different times to interpret those instructions differently. The result is that, despite translated data sets being nominally compatible with the same standard, individual data sets may not be interoperable with each other. The solution is to use the same mappings at each site.

Omission occurs when the standard developers did not foresee a use case when the standard was published, and before some of the data needed to be represented by the data model are known. Most standards provide guidelines for extension to the original data model standard. For example, FHIR provides an Extension resource and OMOP reserves a range of unique identifiers for concept code extensions (2,000,000,000 and over). However, even when extensions are created according to the guidelines, the flexibility in the extension means that the data sets at each site are often incompatible with each other.

For example, if two sites Site 1 and Site 2 both need to represent a novel virus (X) and a new surgical procedure (Y), each of the sites might assign different concept codes to each, as shown in Table 3 below. The solution is to use the same extension codes for the same extensions at each site.

TABLE 3

| | Concept ID assigned | Concept ID assigned |
|---|---|---|
| | in Site 1 | in Site 2 |
| Novel virus X | 2,000,000,001 | 2,000,000,002 |
| New surgical procedure | 2,000,000,002 | 2,000,000,003 |
| Y | | |

As both sites are compatible with the standard but not with each other, the data model in each site is referred to as a ‘dialect’ of the standard. The standard is analogous to a language and data from different sites of origin are incompatible because different sites use a different dialect of that language.

Ambiguity and Omission both creep into documentation of the standard, often when authors of the standard fail to predict every data set that might need to be transformed, and the unique translation decisions that such datasets require. Recent studies show that two systems using the same standard can be only 30-60% compatible due to a combination of these reasons. See, for example, Elmer V Bernstam et al., “Quantitating and assessing interoperability between electronic health records”, Journal of the American Medical Informatics Association, Volume 29, Issue 5, May 2022, Pages 753-760, https//doi. org/10.1093/jamia/ocab289.

Regardless of the cause, the effect is that each site develops its own site-specific dialect. This phenomenon is so common that the formation of dialects occurs nearly every time a new data set is standardised to a common data model, usually without anyone realising until after the data has already been translated. Extra care needs to be taken by transformers, and a protocol needs to be agreed upon, as a precaution.

Some embodiments of the present disclosure provide a method to automate the unification of data translations across multiple collaborating sites, so that the same dialect is formed in all sites. This increases the interoperability among such sites. The method utilises a central repository that is accessible by a translation system at each site. In some embodiments, the central repository is implemented using a computer readable storage medium and computer executable instructions executing on a processor of a computing device coupled to a communications network for communication with one or more sites of origin.

The central repository stores each extension and mapping, and enables the same extensions and mappings to be shared across all sites. Each extension and mapping is associated with a unique identifier in the repository. The method also utilises a translation client present at each site. The translation client is software for execution on one or more processors at the respective site.

The method further utilises an Identifier Issuing Service, which provides a coupling between each site of origin and the central repository. The Identifier Issuing Service is responsible for issuing new unique identifiers to new extensions and mappings, and to reuse existing identifiers for extensions or mappings that are already in the repository.

In some embodiments, the Identifier Issuing Service is implemented using a computer readable storage medium and computer executable instructions executing on a processor of a computing device coupled to a communications network for communication with one or more sites of origin and the central repository.

In some embodiments, the central repository is included within the Repository of Analyses 120, 702, 850, 950, 1020, 1160 described above. In other embodiments, the central repository is implemented as a new network node coupled to a communications network so as to be accessible by all sites of origin within a federated studies network.

In some embodiments, the Identifier Issuing Service is co-located with, or integral to, the central repository, such as by forming part of the Repository of Analyses 120, 702, 850, 950, 1020, 1160 described above. In other embodiments, the Identifier Issuing Service is implemented as a new network node coupled to a communications network so as to be accessible by all sites of origin and the central repository within a federated studies network.

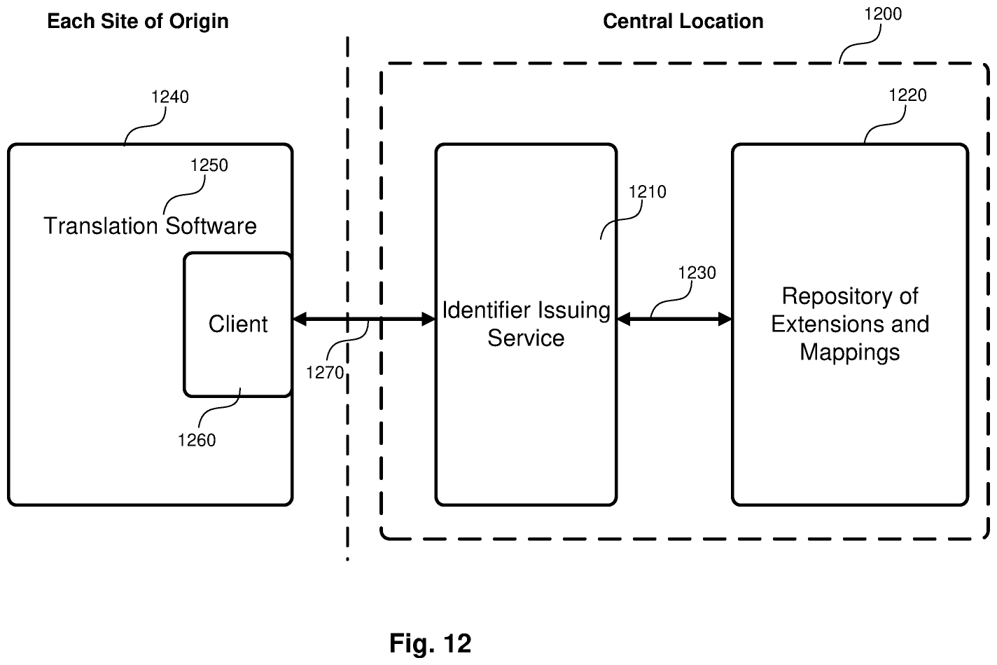

FIG. 12 is a schematic block diagram representation illustrating a portion of a federated studies network having a Site of Origin 1240, which is indicative of any site of origin within a federated studies network. The Site of Origin 1240 includes translation software 1250 and a translation client 1260. The translation client 1260 is configured to communicate, via a first communications network 1270, with an Identifier Issuing Service 1210. The Identifier Issuing Service 1210 is coupled, via a second communications network 1230, to a central repository 1220.

Depending on the implementation, the first communication network 1270 and the second communication network 1230 can be the same communication network, such as a local area network (LAN), a wide area network (WAN), a telecommunications network, or any combination thereof. A telecommunications network may include, but is not limited to, a telephony network, such as a Public Switch Telephony Network (PSTN) or a cellular mobile telephony network, the Internet, or any combination thereof. In other implementations, the first communication network 1270 and second communication network 1230 can be direct physical links, such as a computer bus, in circumstances wherein two or more of the site of origin 1240, the Identifier Issuing Service 1210, and the central repository 1220 are co-located or integrated with each other.

In the example of FIG. 12, the Identifier Issuing Service 1210 and the central repository 1220 are shown as forming part of a Central Location 1200. However, it will be appreciated that the Identifier Issuing Service 1210 and the central repository 1220 can be separate nodes positioned remotely from each other, co-located proximal to each other, or even integral with each other in different embodiments of the present disclosure.

The online central repository stores and retrieves data translation elements (as described below) for a system, as well as unique identifiers for defined extensions. The central repository utilises the unique identifiers to retrieve requested extensions. The central repository can be specific for these elements or more generally supporting other elements as well, such as federated analysis workflows. Separate repositories can be used for each element, as long as the various repositories are together logically equivalent to a single repository.

The central repository acts as a storage facility that stores and retrieves data translation elements, but does not execute the data translation elements. The translation systems residing on each site of origin use the elements during the translation process. Depending on the nature of the data translation elements and the particular application, some of the data translation elements may be executed, some may be used as references, and some may be used as resources by the translation system.

The Identifier Issuing Services ensures that all sites refer to the same translation element using the same identifier. The repository allows all translators at each site of origin to download, via translation clients, the same translation element by the unique identifier associated with that particular translation element.

The result is that all translation systems at the respective sites of origin refer to the same element with the same identifier, as well as using the same element that each translation system retrieves from the central repository. The translation elements can only be used by the translators at the sites of origin, because the translation occurs on patient data, and patient data does not leave its site of origin.

The translation client 1260 is used by each translation system in each site of origin, such as the data model translator 114 of FIG. 3, to share and retrieve a set of data translation elements with each other. When the translation client 1260 identifies a need for an extension, the translation client 1260 registers the need with the Identifier Issuing Service 1210, requesting a unique identifier for the new extension. The Identifier Issuing Service 1210 sends a query to the Central Repository 1220 to check whether the requested extension already exists.

When the requested extension exists, the Central Repository returns the requested identifier associated with the existing extension to the Identifier Issuing Service 1210, which in turn forwards the associated identifier to the translation client 1260.

When the requested extension does not exist in the Central Repository, the Identifier Issuing Service 1210 issues a new unique identifier for the requested extension to the translation client 1260.

In some embodiments, the set of data translation elements includes, but is not limited to, one or more of the following:
Novel Concepts
Value Sets
Novel Concept Relationships
Novel Resources
Data Quality Tests
Population Characterisation Scripts
Field Mappings
Concept Mappings

Novel Concepts are short phrases used in the data being transformed to ascribe meaning to data where such phrases do not already exist. Concepts are routinely used in healthcare data to provide common representations for diagnoses, medications, procedures and other clinical concepts, as well as meta-data.

Many terminologies exist, and some comprise millions of phrases, but during the course of mapping a dataset it is still possible for new phrases to be required. For example, when a new pathogen is discovered it may require a new unique name, so that this name can be referred to in a diagnosis or a laboratory finding. Novel therapies require novel descriptions as a way to refer to those therapies accurately in data. If data records in multiple sites contain references to such a novel virus or therapy, then a Novel Concept is needed to ensure that the references to the particular virus, therapy, etc. are consistent and the particular virus, therapy, etc. are represented in the same way across all sites and thus all data sets.

Value Sets (which may also be referred to as Concept Sets) are named groups of concepts. While typically Value Sets are not part of a data model or a data set, Value Sets are often used as references in the translation process, and in other elements, such as Data Quality Control tests and Population Characterisation Scripts (see below). Therefore, variation in Value Sets can lead to the formation of different dialects. Accordingly, so ensuring that all sites use the same Value Sets is important for ensuring consistency across data sets.

Novel Concept Relationships are relationships between two or more elements described by existing or novel concepts. Relationships between concepts and other concepts are often used in translation to find the best translation candidate when a perfect translation does not exist, such as from “Fracture of neck of femur” to “Neck of femur structure”. For example, one standard terminology might not differentiate between different fractures, so a phrase like “fracture of tibia” might be translated to “fracture” as the closest, more generic alternative. In this case, the relationship between “fracture of tibia” in one terminology and “fracture” in the other may be used to perform the best possible translations. It is also possible to use multiple relationships to traverse from one concept to the next concept when there is no direct relationship between adjacent concepts. Novel Concept Relationships are relationships between a novel concept and an existing concept, between two novel concepts or a new relationship between two existing concepts.

Novel Resources—when a data standard does not have a specific way to represent different kinds of data that are important to multiple sites, a column (or resource) can be created to store that data. For example, OMOP uses a “person” table to record patient information, but the “person” table does not include identifying information such as name and phone number. Novel Resources in the form of additional columns can be added to the person table to hold this additional kind of data. In another example, adding pharmacy inventory data to the OMOP standard would require a Novel Resource in the form of a table. Novel Resources can take other forms apart from the two examples given here. For example, Novel Resources in FHIR have a sub-tree structure.

Data Quality Tests are used before, during, and after the translation process to verify and compare the data before and after translation. Data Quality Tests can take many forms, such as SQL queries or Python scripts, and yield a pass or fail mark. In some embodiments, hundreds of tests are used together to validate the data quality. In some embodiments, the quality of the data is determined by the tests and the aspects of the data covered by the tests, so it is important for multiple collaborating sites to use exactly the same Data Quality Tests. The Data Quality Tests are interpreted and used by translation systems 1250 on respective sites of origin.

Population Characterisation Scripts are different from Data Quality Tests in that Population Characterisation Scripts do not have a pass or fail mark. Population Characterisation Scripts produce a value, a distribution, or some other indication to a particular aspect of the population, such as the proportion of male to female patients in a particular data set. The outputs of Population Characterisation Scripts are used to distinguish variation in the patient population from dialect variation, but only if all sites characterise the population in the same way. The Population Characterisation Scripts are interpreted and used by translation systems 1250 on respective sites of origin.

Field Mappings record how source data was translated. The exact format of mappings depends on both the source and the target. For example, a Field Mapping for data from an electronic healthcare record system to OMOP can have the mapping “Data from the “Encounter” table's “patient” field in the source dataset was mapped to the standard's “visit_occurrence” tables's “person_id” field”. Field Mappings can also be more complex, involving multiple fields and/or IF/ELSE conditions or other logic. Different computer languages can be used to represent Field Mappings. An example of a computer language specific to Field Mappings is LinkML (https://linkml.io/).

Concept Mappings map a phrase or a coded concept from one system into a coded concept from another system. Like Field Mappings, Concept Mappings can be formally represented in different computer languages. A specific language for Field Mapping is the Simple Standard for Sharing Ontology Mappings (SSSOM) (https://mapping-commons.github.io/sssom/).

How each of these elements is represented formally may vary from using snippets of computer languages, like Python or SQL, to specific languages for a particular element type. However, all data translation systems need to be able to parse the element, either directly by using the same language or through another translation service from one formal representation to another.

The Identifier Issuing Service 1210 of FIG. 12 is a component of the system that connects between each site of origin 1240 and the central repository 1220. The primary role of the Identifier Issuing Service 1210 is to ensure that data translation elements with the same semantic meaning have the same unique identifier. The Identifier Issuing Service can search the central repository 1220 and issue new unique identifiers if an equivalent element does not already exist.

As discussed above, when translation software 1250 executing on a site of origin identifies a need for an extension to the standard, the translation software uses the translation client 1260 to send a request to the Identifier Issuing Service 1210 requesting a unique identifier for the new extension. The Identifier Issuing Service 1210 checks whether an equivalent extension already exists within the central repository 1220. If an equivalent element already exists, the Identifier Issuing Service 1210 retrieves the existing identifier from the central repository 1220 and returns the retrieved identifier for that element to the requesting translation client 1260, rather than issue a new one. The translation client 1260 forwards the unique identifier to the translation software 1250.

Alternatively, if an equivalent extension does not already exist in the central repository 1220, the Identifier Issuing Service 1210 generates a new unique identifier for the new extension, such as by using a random number generator or the like, and returns the new unique identifier to the requesting translation client 1260. The Identifier Issuing Service 1210 also transmits the newly generated unique identifier to the central repository 1220 for storage in association with the new extension.

Some implementations of the Identifier Issuing Service can also suggest similar elements that are not identical to the element being searched, using fuzzy-matching, artificial intelligence, or other methods. Some implementations of the Identifier Issuing Service can be integrated as part of the repository.

### INDUSTRIAL APPLICABILITY

The arrangements described are applicable to the research, medical and health industries.

The foregoing describes only some embodiments of the present invention, and modifications and/or changes can be made thereto without departing from the scope and spirit of the invention, the embodiments being illustrative and not restrictive.

Reference throughout this specification to “one embodiment”, “an embodiment,” “some embodiments”, or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

While some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practised without these specific details. In other instances, well-known methods, structures and techniques have not been shown in detail in order not to obscure an understanding of this description.

Note that when a method is described that includes several elements, e.g., several steps, no ordering of such elements, e.g., of such steps, is implied, unless specifically stated.

In the context of this specification, the word “comprising” and its associated grammatical constructions mean “including principally but not necessarily solely” or “having” or “including”, and not “consisting only of”. Variations of the word “comprising”, such as “comprise” and “comprises” have correspondingly varied meanings.

Similarly, it is to be noticed that the term coupled should not be interpreted as being limitative to direct connections only. The terms “coupled” and “connected,” along with their derivatives, may be used. It should be understood that these terms are not intended as synonyms for each other but may be. Thus, the scope of the expression “a device A coupled to a device B” should not be limited to devices or systems wherein an input or output of device A is directly connected to an output or input of device B. It means that there exists a path between device A and device B which may be a path including other devices or means in between. Furthermore, “coupled to” does not imply direction. Hence, the expression “a device A is coupled to a device B” may be synonymous with the expression “a device B is coupled to a device A”. “Coupled” may mean that two or more elements are either in direct physical or electrical contact, or that two or more elements are not in direct contact with each other but yet still co-operate or interact with each other.

As used throughout this specification, unless otherwise specified, the use of ordinal adjectives “first”, “second”, “third”, “fourth”, etc., to describe common or related objects, indicates that reference is being made to different instances of those common or related objects, and is not intended to imply that the objects so described must be provided or positioned in a given order or sequence, either temporally, spatially, in ranking, or in any other manner.

Although the invention has been described with reference to specific examples, it will be appreciated by those skilled in the art that the invention may be embodied in many other forms.

We claim:

1. A computer-implemented federated studies system comprising:
a predefined common data model associated with a study;
a set of sites of origin, each site of origin being associated with: (i) a set of input devices for acquiring study data from subjects, and (ii) a study data device, wherein each study data device includes:
a native database for storing study data associated with the respective site of origin and derived from said set of input devices;
a data model translator for translating stored study data to a format corresponding to said common data model;
a common model database for storing, in said common data model, individual participant values derived from said study data;
a study compute module for determining site aggregate values from said individual participant values; and
an aggregate database for storing said site aggregate values; and
a site of analysis coupled to each site of origin via a communications network, wherein said site of analysis is associated with a study analysis device that includes:
an analysis compute module for processing, in accordance with a predefined analysis profile, site aggregate values received from said sites of origin; and
a reporting module for generating study reports based on said processed site aggregate values;
a central repository for storing and retrieving a set of data translation elements for managing extensions and mappings within study data sets associated with each site of origin; and
an Identifier Issuing Service configured to interface between each site of origin and said central repository to ensure elements within data sets having the same semantic meaning have a common unique identifier.

2. The system according to claim 1, wherein said predefined analysis profile is stored in a repository of analyses associated with said site of analysis.

3. The system according to claim 1, wherein said common data model is selected from the group consisting of: Observational Medical Outcomes Partnership Common Data Model (OMOP CDM), Oracle Health Foundations, National Patient-Centered Clinical Research Network (PCORNet), Sentinel, and Informatics for Integrating Biology & the Bedside (I2B2).

4. The system according to claim 1, wherein said study reports are presented as at least one of: a user interface dashboard for display on a computing device, a printed report, an electronic document, an email, and a webpage.

5. The system according to claim 1, wherein the input devices are adapted to digitally capture data for use in a federated study.

6. The system according to claim 5, wherein the input devices are selected from the group consisting of: electronic health records systems, patient administration systems, medication ordering systems, survey systems, wearable devices, laboratory sensors, imaging machines, electronic thermometers, weather data systems, and computer-implemented data entry devices.

7. The system according to claim 1, wherein the predefined analysis profile defines a set of computer-implementable instructions for analysing a set of data.

8. The system according to claim 1,
wherein each study data device is a computer-implemented study data device coupled to said respective set of input devices;
wherein said study analysis device is a computer-implemented study analysis device; and
further wherein each study data device and said study analysis device are coupled utilising at least one communications link.

9. The system according to claim 8, wherein at least one of said study data devices is implemented in a cloud-computing environment.

10. The system according to claim 1, wherein the federated studies system is implemented using at least one of a computing device or a distributed cloud-based architecture.

11. The system according to claim 1:
wherein each said data model translator includes a translation client configured to communicate with the Identifier Issuing Service when the respective data model translator determines a need for an extension,
wherein said Identifier Issuing Service, on receipt of a request for an extension from a translation client:
communicates with said central repository to determine whether the requested extension exists;
when the requested extension exists, the Identifier Issuing Service retrieves the requested extension from the central repository and forwards the requested extension to the requesting translation client, and
when the requested extension does not exist, the Identifier Issuing Service:
issues a new unique identifier for the requested extension,
forwards the new identifier for the requested extension to the requesting translation client, and
forwards the new identifier for the requested extension to the central repository for storage.

12. A method of conducting a federated study across a plurality of Sites of Origin and using a Site of Analysis, the method comprising the steps of:
defining a common data model for the study;
defining a set of analysis instructions for the study;
at each Site of Origin:
acquiring, utilising least one input device, study data from a set of subjects;
translating said study data to generate individual participant values stored in said common data model;
processing said individual participant values to generate a set of site aggregate values;
transmitting said site aggregate values to said site of analysis; and
processing study aggregate values received from said Site of Analysis in accordance with said defined set of analysis instructions; and
at said Site of Analysis:
processing site aggregate values received from at least one of said Sites of Origin in accordance with said defined set of analysis instructions; and
generating a study report based on said processed site aggregate values;
wherein the method unifies data translations across said plurality of Sites of Origin by:
storing in a central repository a set of data translation elements for managing extensions and mappings within the federated study;
on receipt of a request for a new extension, an Identifier Issuing Service communicating with said central repository to determine whether the requested extension exists;
when the requested extension exists, the Identifier Issuing Service retrieving the requested extension from the central repository and forwarding the requested extension to the requesting translation client, and
when the requested extension does not exist, the Identifier Issuing Service:
issuing a new unique identifier for the requested extension,
forwarding the new identifier for the requested extension to the requesting translation client, and
forwarding the new identifier for the requested extension to the central repository for storage.

13. The method according to claim 12, wherein the method comprises the further step, at each said Site of Origin, of:
generating a report based on said processed site aggregate values and individual participant values.

14. The method according to claim 12, wherein said analysis instructions include a predefined analysis profile.

15. The method according to claim 14, wherein the predefined analysis profile defines a set of computer-implementable instructions for analysing a set of data.

16. The method according to claim 15, wherein said set of computer-implementable instructions include:
definitions of each of the site aggregate values and study aggregate values,
arithmetic and data manipulation operations used to calculate said each of said site aggregate values and said study aggregate values,
instructions for transmission of site aggregate values from sites of origin to the site of analysis,
instructions for receiving site aggregate values from the sites of origin by the site of analysis,
instructions for transmission of study aggregate values from the site of analysis to the sites of origin,
instructions for receiving study aggregate values from the site of analysis by the sites of origin,
instructions for the order of said data manipulation, transmission and reception instructions, and
composition of a report for each one of said Sites of Origin and said Site of Analysis.

17. The method according to claim 12, wherein said common data model is selected from the group consisting of: Observational Medical Outcomes Partnership Common Data Model (OMOP CDM), Oracle Health Foundations, National Patient-Centered Clinical Research Network (PCORNet), Sentinel, and Informatics for Integrating Biology & the Bedside (I2B2).

18. The method according to claim 12, wherein said study report is presented as at least one of: a user interface dashboard for display on a computing device, a printed report, an electronic document, an email, and a webpage.

19. The method according to any claim 12, wherein the input devices are adapted to digitally capture data for use in a federated study, and wherein each Site of Origin includes a translation client configured to transmit a request for a new extension upon detection of a need for a new extension to at least one of the common data model or the set of analysis instructions.

20. The method according to claim 19, wherein the input devices are selected from the group consisting of: electronic health records systems, patient administration systems, medication ordering systems, survey systems, wearable devices, laboratory sensors, imaging machines, electronic thermometers, weather data systems, and computer-implemented data entry devices.
